(19) United States
(12) Patent Application Publication
(10) Pub. No.: US 2021/0313042 A1
(43) Pub. Date: Oct. 7, 2021

(54) System for Generating Treatment Plans for Psychological Illness and Disorders

(71) Applicant: Koa Health B.V., Barcelona (ES)

(72) Inventor: Johan Lantz, Barcelona (ES)

(21) Appl. No.: 17/350,346

(22) Filed: Jun. 17, 2021

Related U.S. Application Data
(63) Continuation-in-part of application No. PCT/EP2019/085763, filed on Dec. 17, 2019.

(30) Foreign Application Priority Data
Dec. 19, 2018 (EP) 18382945.6
Dec. 17, 2019 (EP) PCT/EP2019/085763

(51) Int. Cl. G16H 20/70; G16H 10/60

(52) U.S. Cl. CPC G16H 20/70; G16H 10/60

(57) ABSTRACT

A method for generating a treatment plan for a psychological disorder uses both static and dynamic content to customize the logical model of the treatment plan for a particular patient. A file containing static content is generated at a static layer of a computer program. The file uses a machine readable format to define the treatment plan. A logical model that the program uses to implement the treatment plan is generated at a data modeling layer based on the static content. Events based on responses from the patient that were elicited in response to the static content are generated at an application logic layer. Dynamic content is created using the events. The logical model is then modified based on the dynamic content. The treatment plan is presented to the patient by a visualization layer based on the modified logical model. Responses to the treatment plan are received from the patient.

### CROSS REFERENCE TO RELATED APPLICATION

This application is filed under 35 U.S.C. § 111(a) and is based on and hereby claims priority under 35 U.S.C. § 120 and § 365(c) from International Application No. PCT/EP2019/085763, filed on Dec. 17, 2019, and published as WO 2020/127355 A1 on Jun. 25, 2020, which in turn claims priority from European Application No. EP18382945.6, filed in the European Patent Office on Dec. 19, 2018. This application is a continuation-in-part of International Application No. PCT/EP2019/085763, which is a continuation of European Application No. EP18382945.6. International Application No. PCT/EP2019/085763 is pending as of the filing date of this application, and the United States is an elected state in International Application No. PCT/EP2019/085763. This application claims the benefit under 35 U.S.C. § 119 from European Application No. EP18382945.6. The disclosure of each of the foregoing documents is incorporated herein by reference.

### TECHNICAL FIELD

The present invention has its application within the telecommunication sector, and specifically relates to the deployment of telecommunication network tools, such as applications in mobile user terminals, smartphones, tablets, webpages, etc., that work systematically with a user/patient to improve his mental condition, with or without the direct interaction of a therapist, by treating mental illness and disorders.

The invention involves a technical framework that can be used to design, evolve and deploy treatment programs informatically as well as to collect and evaluate the patient's progress inside the programs over time. The disclosure describes a system and method for generating a computer program or application to build treatment plans for psychological illness and disorders.

### BACKGROUND

Developing a single computer-program based system or mobile application (app) focused on one mental disorder can easily take a team of engineers over a year to complete, and that is just for one specific case. This means that even if focusing only on primary disorders such as depression and anxiety, the time to market for any single treatment plan or app is extremely time consuming and costly. It also has the consequence that less frequent disorders, which cause an equal or even larger strain on individuals and society, will end up last in the priority queue with only a small chance of ever reaching the hands of end users.

The main problem with an app or computer-program based approach is that each program must be addressed based on its unique challenges and conditions. In turn, this leads to an approach where each disorder requires its own dedicated app, which is difficult and costly to be scaled.

Another problem with an app or computer-program based approach is that the engineers building the system do not speak the same language as the doctors needed to design the content of the system. Consequently, reaching a common ground and understanding each others' challenges takes time and is impossible to scale. Each team of engineers and each doctor faces the same initial challenges, increasing the project cost and reducing the chances that the project will launch, will be maintained or can evolve.

U.S. patent application publication 2008/0103833 discloses a system for populating patient records using an evidence-based relational database that compares the medical practitioners' diagnoses to predetermined responses in order to produce accurate patient chart notes. The system integrates stored and generated data into clinical and administrative medical record keeping and billing. Episodic encounters are developed into cases for a specific patient under the care of a practitioner. The subjective symptoms from the patient and the objective observations of the care provider concurrent with the episode are used to generate a diagnosis and a treatment regimen from the evidence-based relational database. The system populates medical and administrative templates. Patient history and updated information are retained in the database and “best practice” treatment plans are continually placed in the relational database from practice guides and experts in the field. US2008/0103833 focuses on the electronic handling of patient records and the generation of such records in an automatic fashion, being more directed to the storage and collection of existing information. But the system does not relate to treating mental problems.

U.S. Pat. No. 6,289,513 focuses on natural language generation and discloses a system for generating an application using a plurality of components, each component defining an application block. US6289513 describes a plurality of user-defined application-specific properties, each property being associated with one of the plurality of components. The system stores non-programmatic user input and receives structured data input via a questionnaire using the plurality of components. The received inputs are data preferably entered via a hierarchical questionnaire data input mechanism, which does not involve predefined lists. The structured questionnaire is converted into text. The text is generated based on the structured data, including dynamic runtime generation of a plurality of simple sentences from a plurality of sub-sentence segments based on user input. The system generates an application based on some of the plurality of user-defined application-specific properties and on the associated components. However, although texts are automatically generated when the user answers a query, the texts do not reference the answers between exercises or questionnaires. U.S. Pat. No. 6,289,513 is focused on building readable content based on the user input, but does not tailor or modify a patient's treatment plan based on the user's selections. U.S. Pat. No. 6,289,513 does not disclose generating new cards or injecting new options in lists.

Therefore, there is a need in the state of the art for developing effective treatment plans of psychological illness or disorders that are easily replicated and scaled.

### SUMMARY

A system and method for generating treatment plans of psychological illness and disorders is implemented based on four hierarchical program layers, including a static layer, a data modeling layer, an application logic layer and a visualization layer. The system includes a content editor, a content storage database and a user interface. At the static layer of a computer program, the content editor generates a file containing static content. In a machine readable format, the file declares a treatment plan defined by a therapist for a patient. The data modeling layer of the program reads the static content of the file and, based on the static content, generates a logical model with which the computer program operates. The application logic layer of the program generates events associated with the patient's inputs that were triggered based on the static content. The events are used to create dynamic content, which is stored in the content storage database. The dynamic content is used to modify the logical model created at the data modeling layer. The visualization layer controls a user interface that presents the logical model of the treatment plan to the patient and receives responses and feedback from the patient.

A computer-implemented method for generating a treatment plan for a psychological illness or disorder uses both static content and dynamic content to modify and customize the logical model of the treatment plan for a particular patient. A file containing static content is generated at a static layer of a computer program. The file uses a machine readable format to define the treatment plan, which is conceived by a therapist for the patient. A logical model that the program uses to implement the treatment plan is generated at a data modeling layer of the program based on the static content. Events based on responses from the patient that were elicited in response to the static content are generated at an application logic layer of the program. Dynamic content is created using the events. The logical model is then modified based on the dynamic content. The treatment plan is presented to the patient by a visualization layer of the program based on the modified logical model. Responses to the treatment plan are then received from the patient.

The file containing the static content is generated by declaring a program block and a program card using the machine readable format of the file. The program block encapsulates a series of exercises of the treatment plan, which are defined by the therapist to elicit responses from the patient. The program card includes the static content and implements a first exercise of the series of exercises to elicit a response from the patient. The program card includes an attribute used by the visualization layer of the program to present the treatment plan to the patient based on the logical model.

A system for generating a treatment plan for a psychological illness or disorder includes a content editor, four hierarchical layers of a computer program, a content storage database, and a user interface. The static layer of the program is implemented on a processor. The data modeling layer of the program is implemented on the processor and is built on the static layer. The application logic layer of the program is implemented on the processor and is built on the data modeling layer. The visualization layer of the program is implemented on the processor and is built on the application logic layer.

The content editor of the program is implemented on the processor and generates, at the static layer, a file containing static content. The static content defines the treatment plan using a machine readable format. The treatment plan is conceived by a therapist for a particular patient. The data modeling layer creates a logical model based on the static content in the file. The program uses the logical model to implement the treatment plan. The application logic layer generates events based on responses from the patient elicited in response to the static content. The events are used to create dynamic content, which in turn is used to modify the logical model. The processor is configured to create the dynamic content using the events associated with the responses from the patient elicited in response to the static content included in a program card and stored in the content storage database. The user interface is used by the visualization layer to present the treatment plan to the patient based on the logical model. The user interface is also used to receive responses from the patient.

The content editor uses the machine readable format to generate the file at the static layer by declaring the program card and a program block that encapsulates a series of exercises of the treatment plan. The series of exercises is defined by the therapist to elicit responses from the patient. The program card implements a first exercise of the series of exercises to elicit a response from the patient. The program card includes the dynamic content created using the events associated with the responses from the patient, and the application logic layer uses the program card to modify the logical model and to customize the treatment plan to the particular patient. The program card includes an attribute used by the visualization layer of the program to present the treatment plan to the patient based on the modified logical model.

Other embodiments and advantages are described in the detailed description below. This summary does not purport to define the invention. The invention is defined by the claims.

### BRIEF DESCRIPTION OF THE DRAWING

The accompanying drawings, where like numerals indicate like components, illustrate embodiments of the invention.

FIG. 1 shows a schematic high level structure of a system for generating a treatment plan for patients of psychological illness and disorders, according to an embodiment of the invention.

FIG. 2 shows an example of exercise reuse across treatment programs.

FIG. 3 shows an outline of a disorder program description in JavaScript Object Notation (JSON) format.

FIG. 4 is a table of the elements of one embodiment of a disorder program.

FIG. 5 is a table of the contents of a block of one embodiment of a disorder program.

FIG. 6 is a table listing examples of the contents of an Exercise element of a block.

FIG. 7 is a table listing examples of the contents of a Card element of a block.

FIG. 8 shows a user interface with a text area generated for a card for collecting free-text input by a patient.

FIG. 9 shows a portion of the disorder program description in JSON format declaring an array of textAreas for user input.

FIG. 10 shows a portion of the disorder program description in JSON format declaring a static checklist of patient questions.

FIG. 11 shows a user interface with a visualization of content for a treatment program statically declared as a checklist.

FIG. 12 shows a portion of the disorder program description in JSON format that assigns a unique identifier to a static checklist.

FIG. 13 shows a portion of the disorder program description in JSON format that generates a patient checklist.

FIG. 14 shows a user interface with a visualization of a dynamically generated card where the patient selects two options.

FIG. 15 shows a user interface with a visualization of a first dynamically generated card based on a selected first option.

FIG. 16 shows a user interface with a visualization of a second dynamically generated card based on a selected second option.

FIG. 17 shows a portion of the disorder program description in JSON format that calls an array of checklists in a card inside an exercise.

FIG. 18 shows a user interface with a visualization of a static card with dynamically injected checklist options for a second checklist.

FIG. 19 shows a portion of the disorder program description in JSON format that uses filtering parameters to determine whether the hide cards.

FIG. 20 shows a user interface with a visualization of replies injected into a generated card.

FIG. 21 shows a portion of the disorder program in which the declaration of a card generates dynamic lists from the patient's answers in previous exercises.

FIG. 22 shows a portion of the disorder program in which a unique identifier is marked using the three angle brackets [[[ ]]].

FIG. 23 shows a flow diagram of a novel method for generating a treatment plan for patients of psychological illness and disorders.

### DETAILED DESCRIPTION

Reference will now be made in detail to some embodiments of the invention, examples of which are illustrated in the accompanying drawings.

The matters defined in this detailed description are provided to assist in a comprehensive understanding of the invention. Accordingly, those of ordinary skill in the art will recognize that variation changes and modifications of the embodiments described herein can be made without departing from the scope and spirit of the invention. Also, description of well-known functions and elements are omitted for clarity and conciseness.

The embodiments described below can be implemented in a variety of architectural platforms, operating and server systems, devices, systems, or applications. Any particular architectural layout or implementation presented herein is provided for purposes of illustration and comprehension only and is not intended to limit aspects of the invention.

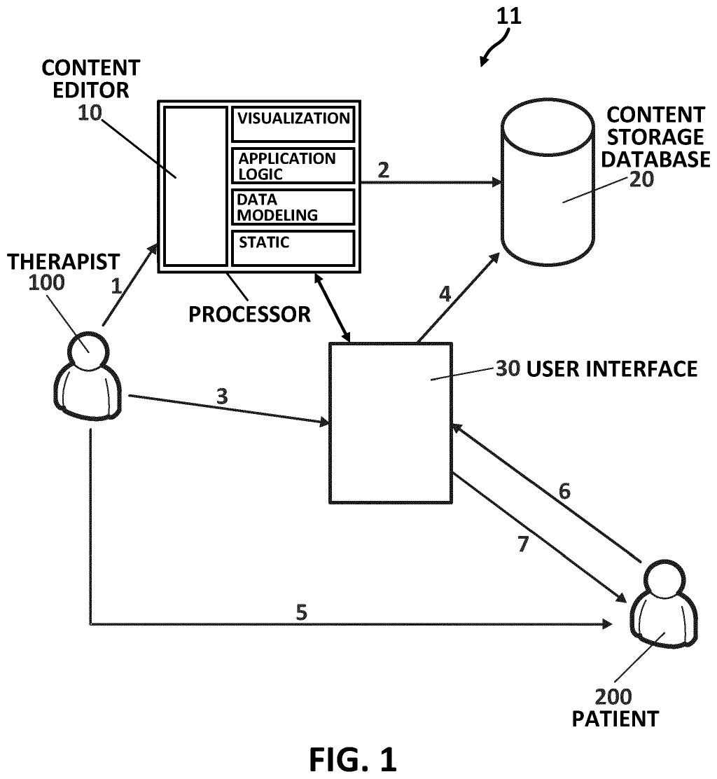

FIG. 1 is a schematic diagram of a novel system 11 for developing treatment plans that includes a content editor 10, a content storage database 20, an administrator interface panel or user interface 30. The system 11 is used by a therapist 100 to develop a treatment plan for a patient 200. The system 11 solves the aforementioned problems and overcomes previously explained state-of-art work limitations.

The system 11 provides a way to build a framework for developing clinical and non-clinical treatment plans in the form of computer programs, based on a generic approach that can be reused to a large extent between different types of mental illnesses and disorders.

The system 11 allows for a complete isolation and externalization of the program content creation, translation, etc., by fully separating the technical side of developing the App or computer program from the clinical side of developing the treatment plan.

In another embodiment, a novel computer-implemented method for generating treatment plans for psychological illness and disorders includes the steps:

at a static layer of the system 11, generating a file containing data static content that defines, using a machine readable format, a treatment plan created by a therapist for a patient;

at a data modeling layer of the system 11, reading the static content of the file and creating a logical model, based on the static content, with which a computer program works;

at an application logic layer of the system, generating events associated with inputs from the patient that are triggered for the static content defined at the static layer, the events being used to create dynamic content that is either stored or dynamically used to modify the logical model created at the data modeling layer; and

at a visualization layer of the system 11, presenting the logical model to the patient 200 and receiving the inputs from the patient 200.

The novel system 11 is configured to implement the aforementioned method and includes a processor that implements the four layers. The visualization layer is built on the application logic layer, which itself is built on the data modeling layer. The data modeling layer is built on the static layer. The content storage database is connected to the processor. The processor includes a content editor with means for generating, at the static layer, a file containing data static content. The data static content defines a treatment plan using a machine readable format. The therapist 100 creates the treatment plan for the patient 200.

The system 11 also includes a means for reading the static content of the file and for creating, at the data modeling layer, a logical model based on the static content. A computer program operates based on the logical model. The system 11 also includes a means for generating, at the application logic layer, events associated with inputs from the patient 200 that are triggered for the static content defined at the static layer. The events are used to create dynamic content that is either stored in the content storage database 20 or used dynamically to modify the logical model created at the data modeling layer.

The system 11 includes a user interface at the visualization layer, such as the user interface 30, for presenting the logical model to the patient 200 and for receiving the inputs from the patient.

The novel system and method has a number of advantages with respect to conventional methods for developing treatment plans for psychological illness and disorders.

The system 11 provides doctors and experts with a four-layered architecture platform of tools to develop new dynamic treatment plans without the technical barriers and costs of writing software code for dedicated applications for each disease or disorder. With this platform in the hands of experts and doctors all over the world, cutting edge treatments can reach the end-user in a scale and at a speed that is unprecedented.

By introducing the dynamic processing of the static content, the treatment evolves from the linear “textbook” format to a dynamic format that is adapted to the user's input and progress. The system 11 dynamically generates new cards, input options and content based on the patient's answers. The system 11 refers to old answers to create dynamic content with new questions, digging deeper into the patient's problems. The four-layered architecture allows for a very high level of reusability, making the time and cost of going to market with new treatments much faster and cheaper.

FIG. 1 shows an overview of a system 11 for designing and developing treatment plans for psychological illness and disorders. The system 11 is used to perform the novel method described in the numbered steps in FIG. 1 in order to move the power of program creation from the engineering organization to the treating organization and to separate the concerns of the two organizations.

In step 1, the therapist 100 develops a treatment plan for a specific mental disorder using the content editor 10. In step 2, the edited treatment plan that is ready to be used is stored in the content storage database 20. In step 3, the therapist 100 configures an account for the patient 200 using the user interface 30. In step 4, the therapist 100 uses the user interface 30 to fetch from the content storage database 20 a specific treatment plan assigned by the therapist 100 to the new patient 200.

In step 5, the therapist 100 informs the patient 200 about the availability of the treatment plan. For example, the therapist 100 provides the patient 200 with an activation code or a password if the plan is restricted from public access. In step 6, an app or computer program running on the patient's equipment, e.g., smartphone or personal computer, contacts the user interface 30 by providing the activation code or password that identifies the patient 200. In step 7, the user interface 30 responds to the request received from the app or computer program, for example after validating the activation code or password received from the app or computer program within the request. The user interface 30 activates the correct treatment plan for the patient 200. Alternatively, the treatment plan is downloaded to the patient's equipment.

The system 11 includes four data layers: (1) the static layer, (2) the data modeling layer, (3) the application logic layer, and (4) the visualization layer. The static layer is a file with data content defined by a specialist, the therapist 100, using a predefined machine readable format, such as JavaScript Object Notation (JSON) format; Extensible Markup Language (XML), or custom formatting. the therapist 100 uses the file of the static layer to create and edit the treatment plan description.

The data modeling layer reads the static file containing the treatment plan and transforms it into a logical model that a computer program can work with. The application logic layer works with the generic logical models created by the data modeling layer. The application logic layer translates into events the patient actions that are triggered for the content defined at the static layer. The application logic layer either alters the treatment plan dynamically based on the generated events or registers the events to be interpreted by a practitioner or a machine.

The visualization layer presents the models created at the data modeling layer (and potentially modified at the application logic layer) to the patient 200 in a visual way. The visualization layer offers ways for the patient 200 to interact with the model so that actions can be detected and acted upon as described in the application logic layer.

One of the greatest benefits of the novel system 11 results from allowing the same visual design to be used across various apps. Colors and images can naturally change, but the overall features and navigation of the system stay the same. In this scenario, only the program file at the static layer (1) is replaced, but the rest of the layers (2)-(4) stay completely unchanged. This allows the therapist 100 to design a program for an unsupported condition without requiring any engineering efforts.

In one embodiment, the visualization layer is built using visual components, which are isolated software blocks, each focused on representing one part of the user experience. For example, the software blocks can represent anything from a screen with some text to an interactive exercise. A user-facing feature is built from layers of visual components in the visualization layer (4). In one example, a visual component can be a self contained feature. In another example, a visual component “component-c” can be built on another visual component “component-b”, which is in turn built on a visual component “component-a”. For some treatment plans, a more specialized user experience might be required, especially on a higher level where the complete reuse of the visual layer might not be feasible. In such a scenario, partial component reuse is also an option. While the main application flow and user experience might change, the majority of the underlying generic components may still be reused. The layered visual architecture allows for a great level of flexibility at the lowest possible cost. Due to the complete separation of the user-facing features from the rest of the architecture, it is also possible to build a completely different app by replacing all of the visual components in the visualization layer (4) with something completely new. While the user-facing features might require a complete rework and lots of attention, the content of the other layers (2)-(4) can stay unchanged.

Furthermore, because the data modeling layer (2) sets the rules for how the program can be declared, reusing static content in the static layer (1) between programs is an efficient procedure. This means that exercises created for a treatment plan or disorder in program A can be cross referenced from treatment plans or programs for other disorders.

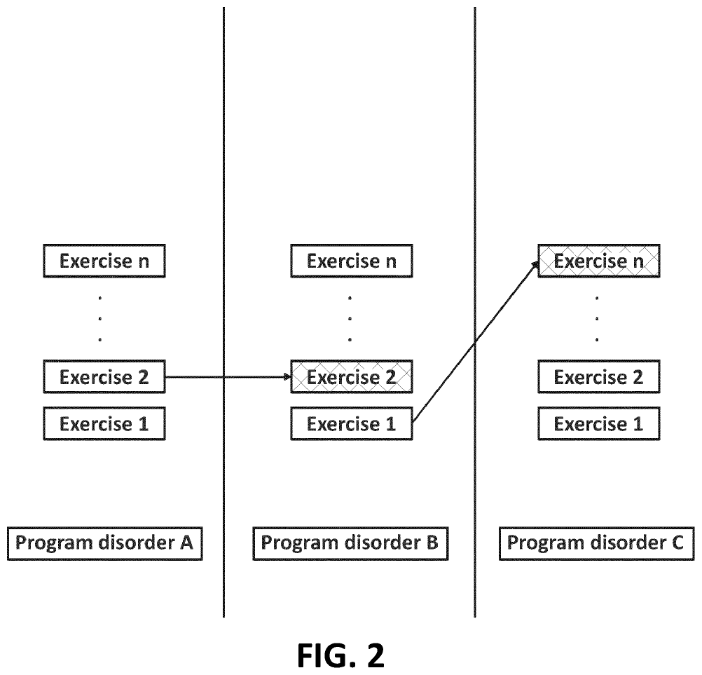

FIG. 2 illustrates the example of how an Exercise 2 is referenced by a program for disorder B even though Exercise 2 was generated for a program for disorder A. Similarly, FIG. 2 illustrates how an Exercise 1 in the program for disorder B is referenced as Exercise n by a program for the disorder C. In this example, the programs A, B and C are each defined for different mental disorders.

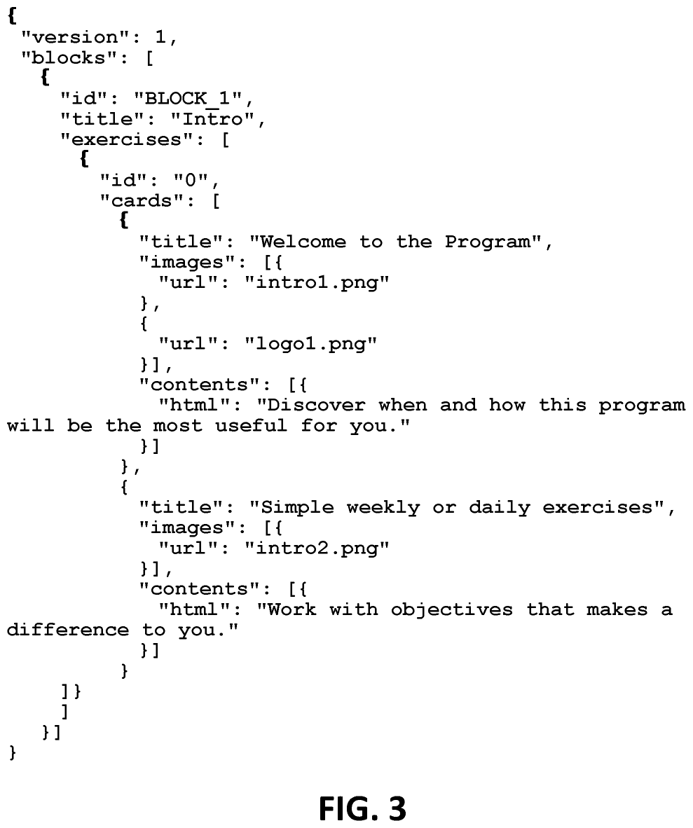

The technical structure of the system 11 mimics a more traditional treatment plan as offered by the therapist 100 and starts off with an encapsulating container called the disorder program. The disorder program is what encapsulates everything that the therapist 100 would offer the patient 200 during months of sessions. A disorder program is focused on talking the patient 200 through a structured and ordered plan for treating a mental condition, such as depression. The outline of a disorder program description could resemble the computer program implemented description shown in FIG. 3, which is in JSON format. In the example of FIG. 3, the disorder program contains only a single Block and a single Exercise.

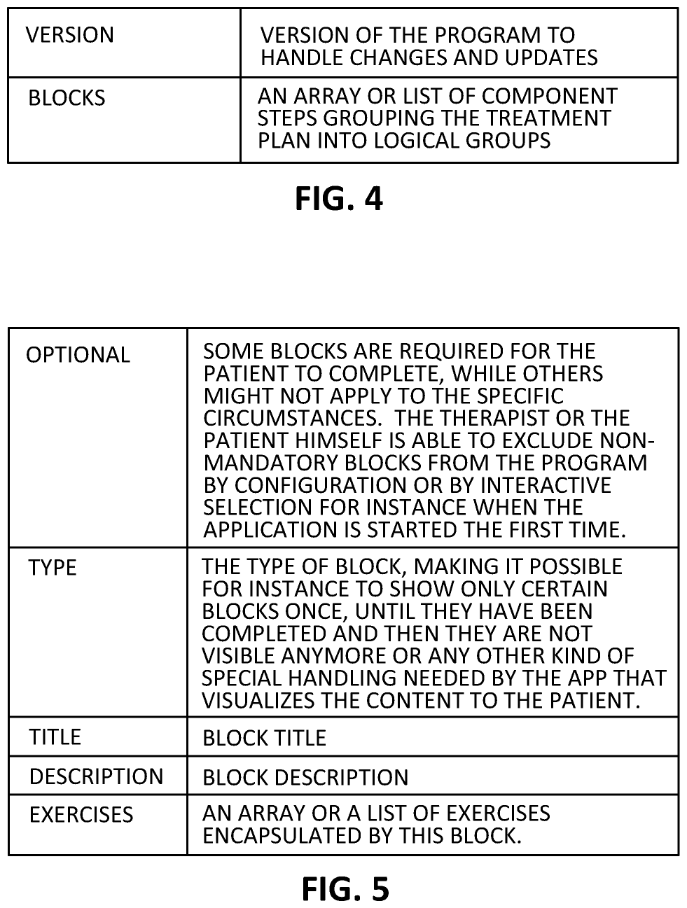

The tree structure of the disorder program of FIG. 3, which is grouped into a Block and then Exercises and Cards, provides the therapist 100 with a well-defined way to create a linear or even nonlinear treatment plan arranged into different steps. The details and exact parameters for each building block of the disorder program can naturally vary over time and with the complexity of the disorder program. One embodiment includes the elements listed in FIG. 4.

A disorder program contains one or more Blocks. A Block is a container for treatment segments that creates a logical group of exercises offered to the patient as part of the treatment. A Block has at least the characteristics listed in FIG. 5.

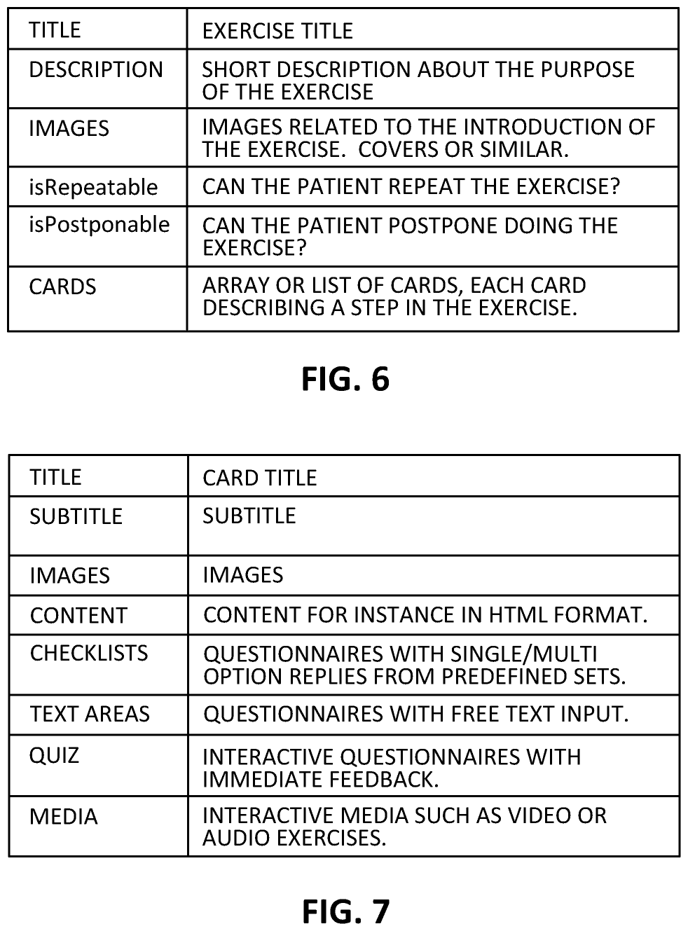

A block typically encapsulates a series of Exercises logically grouped together. For a condition that involves asocial behavior, a typical block could contain exercises gradually encouraging the patient to leave the home. An Exercise has at least the characteristics listed in FIG. 6.

A Card is a generic concept that describes a part of an exercise. An exercise is built from one or more cards, each fulfilling a specific purpose. The high level attributes of a card are similar to the ones for an Exercise or Block. Some examples of the contents of a card are listed in FIG. 7. The example characteristics listed in FIG. 7 are enough to display static information such as an explanation or instructions given to the patient 200. But the listed characteristics provide only a high-level description of an interactive book. The main advantages of using the dynamic approach is that an app can present information in different ways if more advanced cards can be defined in the program file. Some examples of more advanced cards are described below.

Audio cards: As part of step 1 of the novel method for designing treatment plans for psychological disorders, the therapist 100 declares a link to an audio file. When the program is run, the patient 200 is presented with an audio exercise. In steps 2-4, the app is already able to model, interpret and visualize the audio card.

Consequently, while defining the exercise and its corresponding cards, the therapist 100 need only declare on each card which audio properties will automatically render to the user.

Video cards: Video cards are similar to audio cards. The therapist 100 must define one or multiple cards in an exercise as video cards, and the end result for the patient 200 is that a therapeutic video is presented when that step of the Exercise is reached.

Content cards: Content cards are composed of a standard web page containing explanatory texts, images and other static content.

Advanced Cards: The types of cards given as examples above (audio, video, content) solve specific and static use-cases. This goes a long way towards providing the patient with one-way information based on the content in the static layer, but the audio, video and content cards do not provide the interactive and dynamic program output that is needed in order to take the treatment to the next level. In order to do that, the concepts of text areas and checklists in cards are introduced here in order to collect user input. These components are then arranged in the desired way in an advanced card.

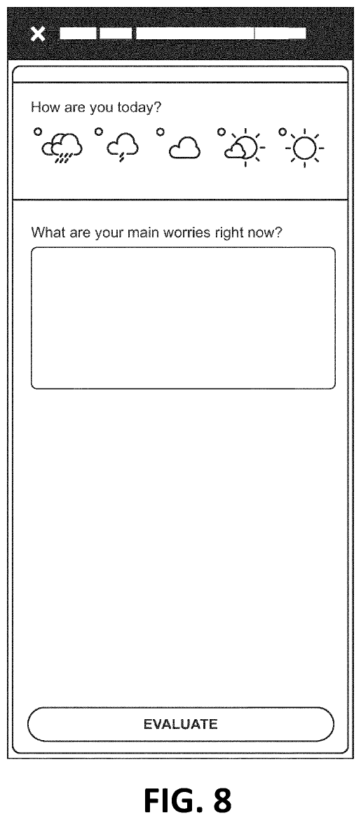

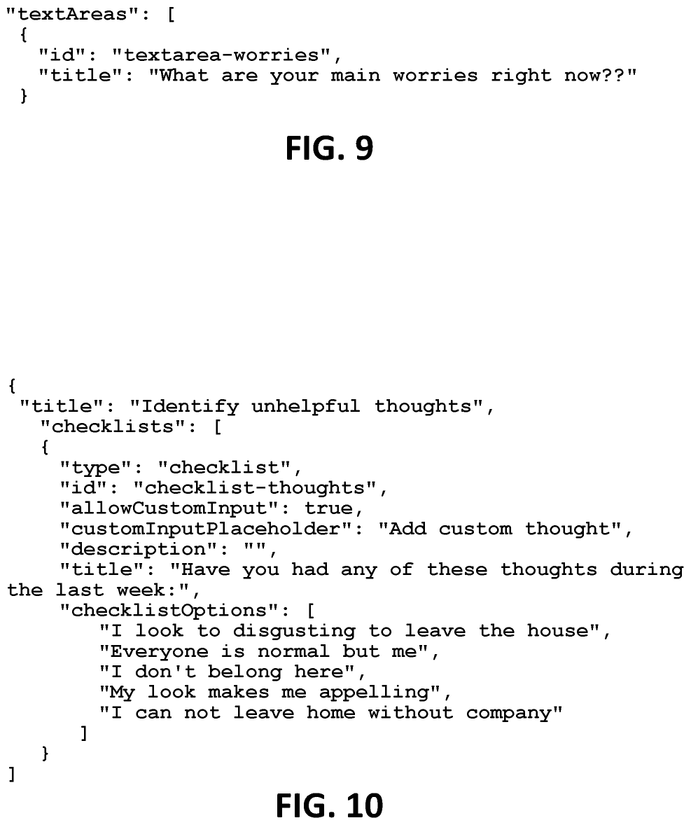

TextAreas: In some exercises, the patient 200 must provide free-text input to the exercise. This is done using a textArea component at the static layer (1), as shown in FIG. 8 (x3) on the user interface of a smartphone. The free-text input is stored with a unique identifier so that the patient's answer can be retrieved later on. The patient's answers can then be used in other exercises, for the patient's self-reflection or for analysis by the therapist. An example of a declaration of an array of textAreas for user input, in JSON format, is listed in FIG. 9.

Checklists: During certain phases or checkpoints in the Program, it is important to collect feedback from the patient 200 or to use the patients' thoughts or reflections as input for future exercises. A straightforward way to collect a patient's input is to display predefined checklists from where the patient 200 can select the best fitting options.

In the simplest scenario, the program file at the static layer (1) declares a static list of options that should be presented to the patient 200 on a given card. The sample card simply has a title and a declaration for the text for the button leading to the next page (nextButtonText), and inside it contains a single checklist asking for user input. An example of a simple static checklist asking the patient 200 about the patient's negative thoughts during the last couple of days is listed in FIG. 10.

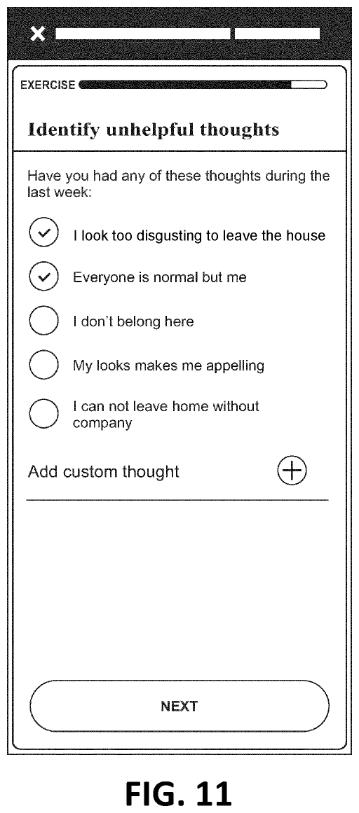

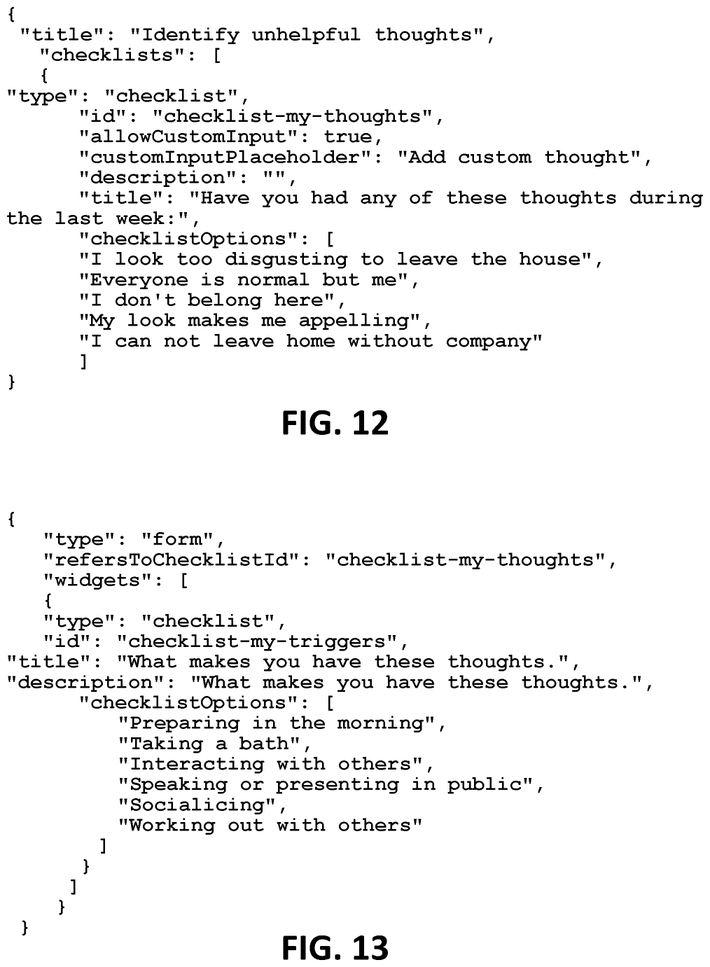

The declaration of the portion of the disorder program listed in FIG. 10 renders into the image visualized by visualization layer (4) onto the user interface of a smartphone as shown in FIG. 11. A checklist where the patient 200 can choose from the various options is statically defined. There are several ways to connect user input across exercises at any point in time during the patient journey through the disorder program. In order to accomplish this, an option of implementation is to uniquely identify previous answers by assigning a unique identifier to the individual checklist. A portion of the disorder program that assigns an identifier to a checklist in order to identify previous answers is shown in FIG. 12.

Thus, when the patient 200 selects from the available options of the checklist shown in FIG. 11, the responses are stored based on the checklist identifier, and the responses can be retrieved and used in future exercises.

With the ability to retrieve previous input of the patient 200, new additional cards can be generated referring back to the replies in a previous exercise. Dynamically generated cards can be declared based on previous patient input as in the checklist declared by the portion of the disorder program shown in FIG. 13.

The program portion that generates this card introduces the “refersToChecklistId” parameter, which is declared in the static layer (1). The parameter declared in the static layer (1) triggers the application logic layer (3) to generate new cards dynamically based on the patient's input from the first card. Each answer from the “checklist-my-thoughts” card generates one dynamic card asking for the triggers of the initial thoughts in this example.

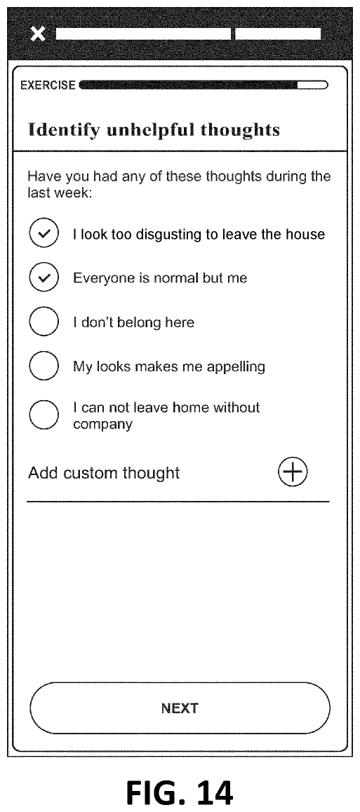

FIG. 14 shows a user interface of a smartphone on which the disorder program generates an image on which the patient has selected two options for the card generator. When processing the card, the application logic layer (3) detects that the card declares the “refersToChecklistId” parameter referencing the generator cards “checklist-my-thoughts”. This triggers the automatic generation and injection of two new cards (one for each option selected) into the deck of cards in the current exercise.

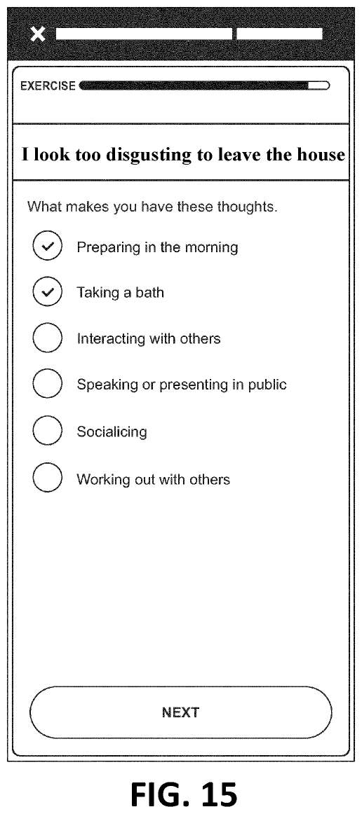

FIG. 15 shows the smartphone interface on which the visualization layer (4) has rendered the first dynamically generated card from the “I look too disgusting . . . ” option selected by the patient in FIG. 14.

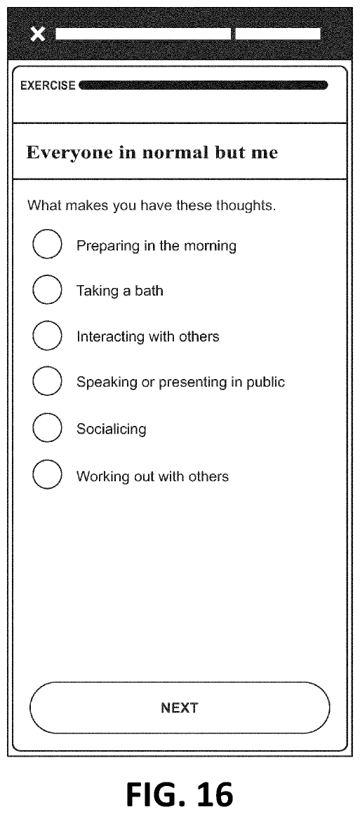

FIG. 16 shows the smartphone interface on which the visualization layer (4) has rendered the second dynamically generated card from the “Everyone is normal . . . ” option selected by the patient in FIG. 14.

Here it is important to notice that only the first “template” for the cards to be dynamically generated is declared in the static layer (1). If multiple answers are provided by the user in the initial questionnaire (checklist-my-thoughts), then multiple dynamic cards are automatically generated. In the event that each dynamic card has a referring title that is the option previously selected, then a static list of new options is used to further drill into the exercise and understand the patient. Also note that the card that declares this behavior in the static layer (1) also assigns an identifier to the new checklist used on the dynamic cards (“checklist-my-triggers”) so as to preserve the answers for future usage. This allows the therapist 100 to dig deeper and deeper by progressively asking only the relevant questions based on previous patient input.

Another important feature of the disorder program that implements the treatment plan is the ability to refer back to previous patient input at certain checkpoints in the program by dynamically generating follow up options. This feature is different from the previously described functionality because the previous answers are provided as a dynamic checklist in order to reflect back as opposed to requiring a new card to be generated based on static options. In order to implement this additional feature, the following steps are taken.

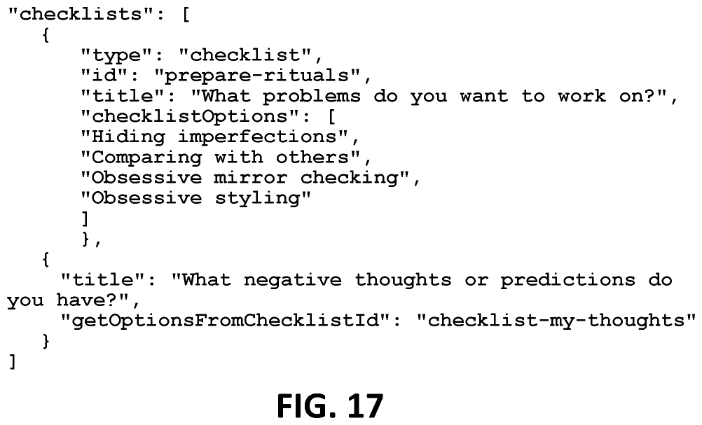

A Card is defined that generates checklist options from previous answers at the static layer (1). The static layer (1) introduces a new parameter called, for example, “getOptionsFromChecklistId”. FIG. 17 shows a portion of the disorder program in which a Card inside an Exercise declares an array of Checklists. Only the checklist array is shown in FIG. 17 for clarity. The first checklist contains a list of static options for the patient 200 to select from. The second checklist, instead of specifying its own options, declares the “getOptionsFromChecklistId” that indicates that the options for this list must be retrieved from previous replies and then injected into the card.

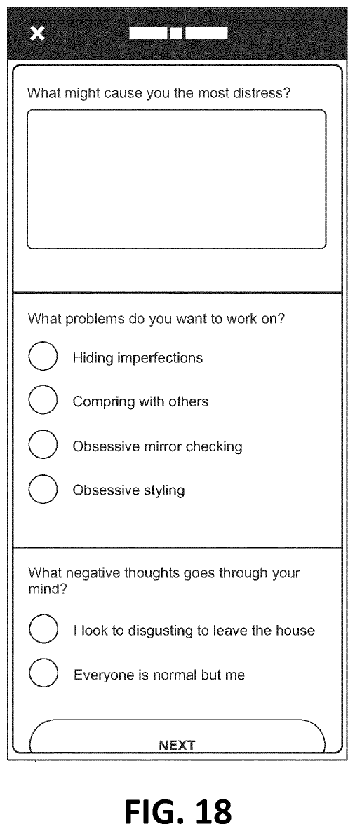

Options in the existing Card are injected at the application logic layer (3). The logic layer (3) dynamically creates and injects checklist options into the questionnaires on the declared Card in order to ask the patient 200 relevant questions following up on previous selections. FIG. 18 shows the smartphone interface with a visualization of how the “getOptionsFromChecklistId” has been used by the logic layer (3) to automatically inject checklist options into a Card. Both checklists from the static layer (1) are visualized as lists of selectable options. The first checklist contains nine static options from the first list declared above. The second list contains the two options that were previously provided by the patient 200 in an earlier exercise and that were stored in “checklist-my-thoughts”.

By automatically injecting previously generated checklists into a Card, the therapist 100 is able to refer back to previous input from the patient 200 and to create follow up questions at different points of the treatment and in different contexts, which cannot be accomplished using a static treatment plan. In this manner, the therapist 100 can determine how the patient's replies to the same questions evolve over time, without requiring any new development from the engineering team. The therapist 100 can refer back to any question at any time.

In some situations, cards with advanced or extensive content must be declared in advanced in the static layer (1). But the these cards are then hidden based on patient input in previous exercises. The cards are hidden because some content might not apply in a current exercise, depending the patient's earlier responses. The appropriate situations for hiding cards can be recognized by using a filter parameter in the static layer (1) that allows the person creating the program to introduce dynamic filtering of the content without the need of any custom development.

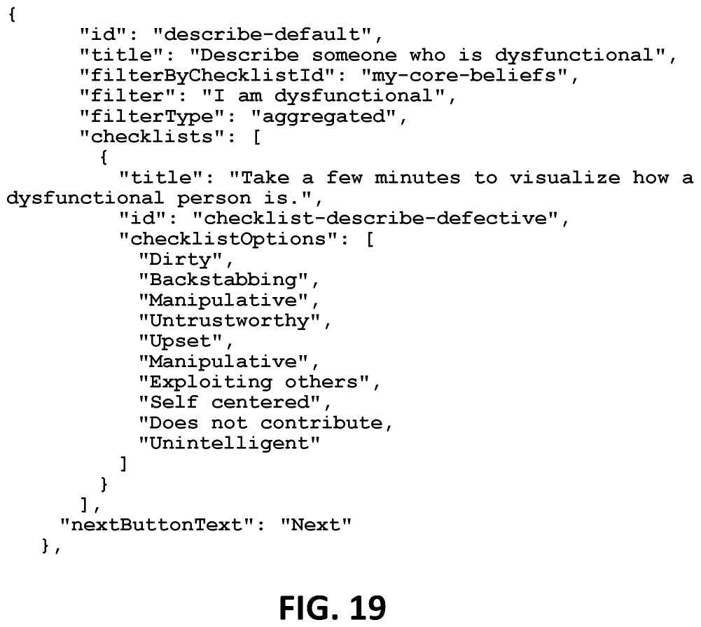

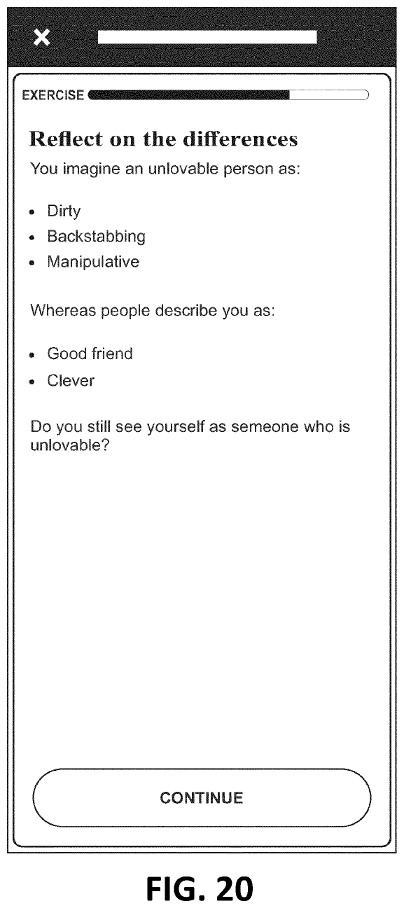

FIG. 19 shows a portion of the disorder program in which three filtering parameters are introduced for the card having the identification “describe-default”. The “filterByChecklistId” refers back to a checklist answered by the patient 200 in a previous exercise. If the “filter” phrase is in the set of answers provided by the patient 200, this card will be shown in order to further work with this issue. If the patient 200, in this case, does not identify himself as defective, the exercise will not focus on further evaluating that belief. Finally, this card uses the “filterType” parameter with the value “aggregated”. This means that all the patient's answers for this exercise over time will be used by the filter as opposed to only the latest replies. This allows the program designer to take into consideration that the patient 200 has repeated the exercise leading up to this one several times and has potentially provided different answers along the progress of the treatment. Aggregating the answers allows the evaluation of all the beliefs compared to just a subset, such as the last ones if needed. FIG. 19 shows an example of a card declaration in the static layer (1) that allows filtering out cards based on previous patient input. FIG. 20 shows a user interface with a visualization of a card generated using the portion of the disorder program shown in FIG. 19 into which replies have been injected.

Dynamic Content Injection

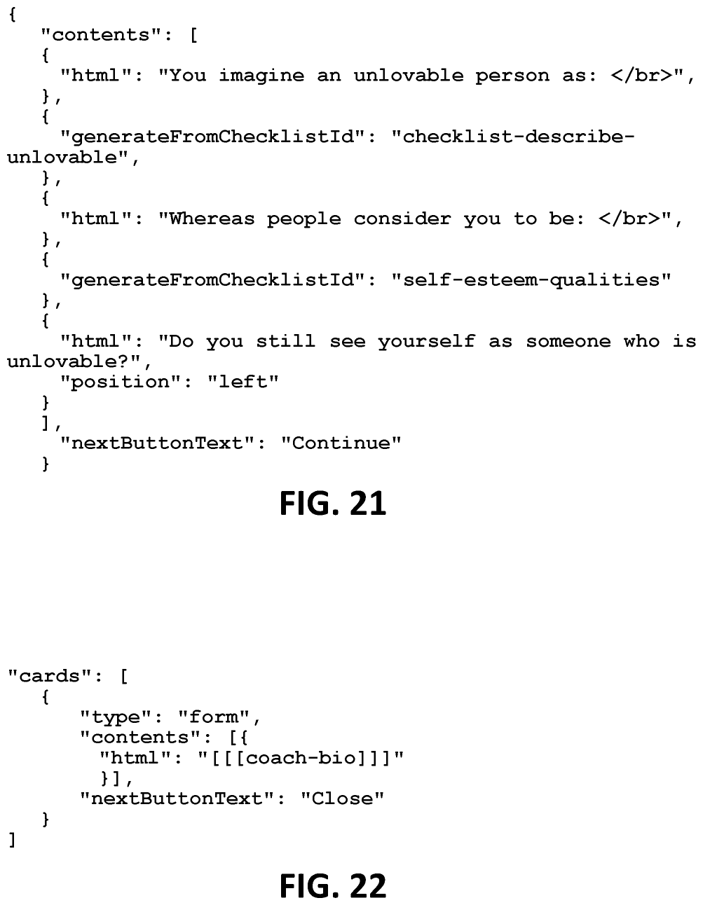

Even though many cards will provide static content to educate the patient 200 about the condition, the content is perceived as much more personalized if replies from the patient 200 are seamlessly injected into the educational cards. This is accomplished using another tag called “generateFromChecklistId” that dynamically creates a visual representation of the replies in a previous exercise and injects the replies into the current card. FIG. 21 shows a portion of the disorder program in which the declaration of a card generates dynamic lists at the static layer (1) from the patient's answers in previous exercises.

Some parts of the content of the cards must change depending on the patient 200 and, for example, the hospital. In one example, the therapist 100 managing the treatment desires the change. In order to give the program a personal and credible feeling, a patient 200 must be assigned to a therapist. This can be a person that has previously treated the patient or someone available only remotely. Either way, the patient 200 needs to be presented with some personal information about the treating therapist 100, such as a photo and a bio. This information cannot be part of the actual treatment program file because not all clients will use the same therapist 100, and the same program can be reused in different hospitals. In order to achieve this personalization, the therapist's information (and other dynamic content) is injected during runtime by the logic layer (3). In order to be able to inject generic dynamic content, modifications are needed on both the static layer (1) and the application logic layer (3).

The novel disorder program uses the concept of a unique tag with a dynamic content identifier. The programming syntax uses three angle brackets and declares the name of the unique identifier inside the brackets. FIG. 22 shows a portion of the disorder program in which the unique identifier is called “coach-bio” and is marked up using the [[[ ]]] brackets. But other embodiments use different tags. FIG. 22 is an example of an exercise that statically declares a single card at the static layer (1) and injects a single generic content element.

In order to be able to read and inject dynamic content, the logical layer (3) introduces a dynamic content storage. The dynamic content storage is a generic read/write repo that has an application programming interface (API) such as the one described below:

setDynamicContent(dynamicContentId: string, content: string) { }

getDynamicContent(dynamicContentId): string { }

The application programming interface allows any part of the application to register dynamic content, for example in html format, to the dynamic content service. Then when setting up the program or when preparing the next card, the application logic layer (3) scans through all content elements to see if there is any content that needs to be dynamically injected. If so, the data in the html tag listed above will be replaced by the content retrieved from the getDynamicContent parameter by passing the unique identifier found in the static program file. The [[[coach-bio]]] tag declared in the static layer (1) is transformed into a visual representation of how the therapist's biographical information is displayed to the patient at the visualization layer (4). The actual html data shown is read from another source which is not related to the program file. This approach allows different data sources to be dynamically combined with the treatment plan without interfering with its content. The therapist 100 is responsible for and can focus on the program creation. The patient 200 knows that a therapist will be assigned to him/her and simply declares in the program file that this information should be shown on a certain page and does not have to worry about the details of how that works. The therapist 100 also does not have to request that additional development work be done. This method of injecting information from tags can naturally be applied to any kind of content that is provided as part of the treatment bundle for the patient, and then the content is injected into the application.

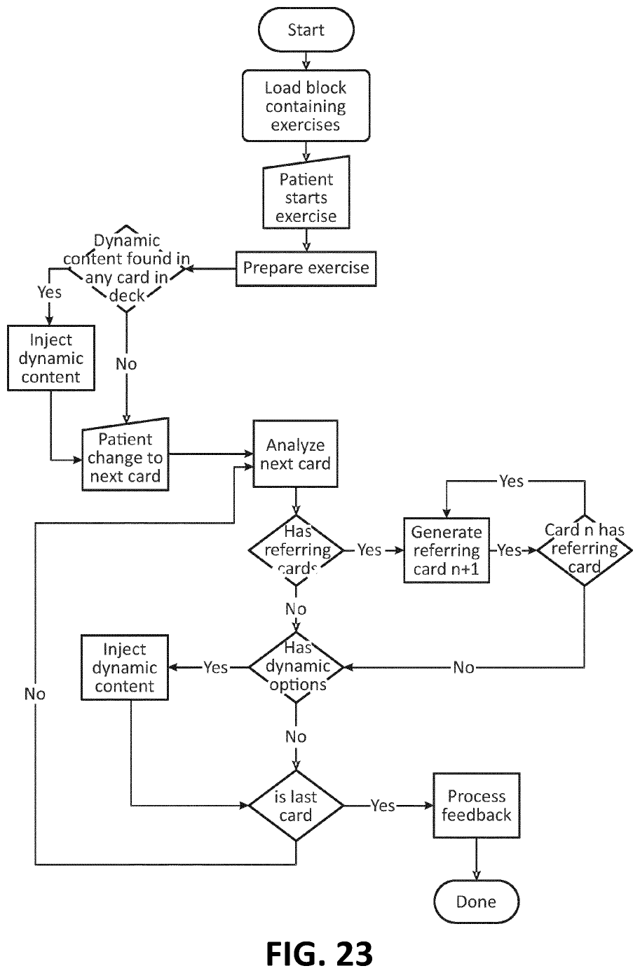

FIG. 23 is a high level flowchart illustrating how the dynamic content of an exercise being loaded by the patient 200 is managed. The program block containing the exercises for a specific treatment plan assigned by the therapist 100 to the patient 200 is loaded in the patient's user terminal (e.g., mobile terminal or personal computer) so that the patient 200 can start one or more of the exercises. In order to prepare an exercise, the patient 200 finds a deck of generated cards and checks whether dynamic content can be found in any card of the deck. If so, the dynamic content is injected into the next generated card; otherwise, the patient 200 is prompted to change from the current card to the next generated card. The next generated card is analyzed to check whether the card is referring to other cards and, if so, all the cards referred to are generated for the exercise. Then, the next generated card is analyzed to check whether the card has dynamic options and, if so, the dynamic options are injected into the card. The process is repeated until the patient 200 is provided with the last card of the deck. Finally, feedback based on the patient's answers is generated.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-18. (canceled)

19. A computer-implemented method for generating a treatment plan for a psychological disorder, comprising:
generating, at a static layer of a program, a file containing static content, wherein the file uses a machine readable format to define the treatment plan, and wherein the treatment plan is conceived by a therapist for a patient;
generating, at a data modeling layer of the program, based on the static content a logical model that the program uses to implement the treatment plan;
generating, at an application logic layer of the program, events based on responses from the patient elicited in response to the static content;
creating dynamic content using the events;
modifying the logical model based on the dynamic content;
presenting, at a visualization layer of the program, the treatment plan to the patient based on the logical model; and
receiving responses to the treatment plan from the patient.

20. The method of claim 19, wherein the generating the file containing the static content involves declaring a program block using the machine readable format of the file, wherein the program block encapsulates a series of exercises of the treatment plan, and wherein the series of exercises is defined by the therapist to elicit responses from the patient.

21. The method of claim 20, wherein the generating the file containing the static content involves declaring a program card using the machine readable format of the file, wherein the program card includes the static content, wherein the program card implements a first exercise of the series of exercises to elicit a response from the patient, and wherein the program card includes an attribute used by the visualization layer of the program to present the treatment plan to the patient based on the logical model.

22. The method of claim 21, wherein the attribute of the program card is a link to an audio file.

23. The method of claim 21, wherein the attribute of the program card is a link to a video file.

24. The method of claim 21, wherein the attribute of the program card is a link to a web page.

25. The method of claim 21, wherein the attribute of the program card is a checklist.

26. The method of claim 21, wherein the creating dynamic content further comprises:
storing the responses from the patient elicited in response to the static content included in the program card; and
declaring an additional program card at the static layer using the machine readable format, wherein the additional program card is used at the application logic layer to modify the logical model, wherein the additional program card includes the dynamic content created using the events associated with the stored responses from the patient, wherein the additional program card includes a second attribute used by the visualization layer of the program to present the treatment plan based on the logical model, and wherein the second attribute is selected from the group consisting of: a link to an audio file, a link to a video file, a link to a web page, a checklist, and a textArea component.

27. The method of claim 19, wherein the machine readable format is selected from the group consisting of: JavaScript Object Notation (JSON) and Extensible Markup Language (XML).

28. A system for generating a treatment plan for a psychological disorder, comprising:
a static layer of a program implemented on a processor;
a data modeling layer of the program implemented on the processor, wherein the data modeling layer is built on the static layer;
an application logic layer of the program implemented on the processor, wherein the application logic layer is built on the data modeling layer;
a visualization layer of the program implemented on the processor, wherein the visualization layer is built on the application logic layer;
a content editor of the program implemented on the processor, wherein the content editor generates at the static layer a file containing static content, wherein the static content defines the treatment plan using a machine readable format, wherein the treatment plan is conceived by a therapist for a patient, wherein the data modeling layer creates a logical model based on the static content in the file, wherein the program uses the logical model to implement the treatment plan, wherein the application logic layer generates events based on responses from the patient elicited in response to the static content, and wherein the events are used to create dynamic content which in turn is used to modify the logical model; and
a user interface used by the visualization layer to present the treatment plan to the patient based on the logical model, wherein the user interface is used to receive responses from the patient.

29. The system of claim 28, wherein the machine readable format used by the content editor is selected from the group consisting of: JavaScript Object Notation (JSON) and Extensible Markup Language (XML).

30. The system of claim 28, wherein the content editor uses the machine readable format to generate the file at the static layer by declaring a program block, wherein the program block encapsulates a series of exercises of the treatment plan, and wherein the series of exercises is defined by the therapist to elicit responses from the patient.

31. The system of claim 30, wherein the content editor uses the machine readable format to generate the file at the static layer by declaring a program card, wherein the program card implements a first exercise of the series of exercises to elicit a response from the patient, and wherein the program card includes an attribute used by the visualization layer of the program to present the treatment plan to the patient based on the logical model.

32. The system of claim 31, wherein the user interface comprises an audio player that plays an audio file linked to the attribute of the program card.

33. The system of claim 31, wherein the user interface comprises a video player that plays a video file linked to the attribute of the program card.

34. The system of claim 31, wherein the user interface comprises a multimedia player that plays multimedia content from a web page linked to the attribute of the program card.

35. The system of claim 31, wherein the user interface comprises a screen that displays a checklist defined by the attribute of the program card.

36. The system of claim 28, further comprising:
a content storage database, wherein the processor is configured to create dynamic content using the events associated with the responses from the patient elicited in response to the static content included in the program card and stored in the content storage database.

37. The system of claim 28, wherein the content editor generates a program card, wherein the application logic layer uses the program card to modify the logical model, wherein the program card includes the dynamic content created using the events associated with the responses from the patient, wherein the program card includes an attribute used by the visualization layer of the program to present the treatment plan to the patient based on the logical model.

38. The system of claim 37, wherein the attribute is selected from the group consisting of: a link to an audio file, a link to a video file, a link to a web page, a checklist, and a textArea component.
